(19) United States
(12) Patent Application Publication
(10) Pub. No.: US 2025/0122477 A1
(43) Pub. Date: Apr. 17, 2025

(54) IMMUNOMODULATING MESENCHYMAL STEM CELLS

(71) Applicant: Global Stem Cell Technology, Evergem (BE)

(72) Inventors: Jan SPAAS, Evergem (BE); Sarah BROECKX, Evergem (BE)

(21) Appl. No.: 18/999,942

(22) Filed: Dec. 23, 2024

Related U.S. Application Data
(62) Division of application No. 17/436,640, filed on Sep. 6, 2021.

(30) Foreign Application Priority Data
Mar. 12, 2019 (EP) 19162270.3

(51) Int. Cl. C12N 5/0775; A61K 35/28

(52) U.S. Cl. CPC C12N 5/0668; A61K 35/28; C12N 2502/11

(57) ABSTRACT

A treatment of an immune-related disease and/or an inflammatory process in an individual by administering a cell composition to a patient, the cell composition including isolated mesenchymal stem cells measured positive for mesenchymal markers CD29, CD44 and CD90 and negative for MHC class II molecules, the cell secretes immunomodulatory prostaglandin E2 cytokine when present in an inflammatory environment or condition.

### CROSS REFERENCE TO RELATED APPLICATIONS

The application is a divisional of U.S. patent application Ser. No. 17/436,640, filed on Sep. 6, 2021, which is a National Phase of International Application No. PCT/EP2020/056625, filed on Mar. 12, 2020, which claims priority to European Patent Application No. 19162270.3, filed on Mar. 12, 2019, the entire disclosures of which are incorporated herein by reference.

### TECHNICAL FIELD

The invention relates to an isolated mesenchymal stem cell and a cell composition comprising said mesenchymal stem cells. Furthermore, the cell composition of the invention can be used in the treatment of immune-related diseases and inflammatory processes to modulate the immune system.

### BACKGROUND

Mesenchymal stem cells (MSCs) are multipotent stem cells characterized by self-renewal, production of clonal cell populations, and multilineage differentiation. They exist in nearly all tissues and play a significant role in tissue repair and regeneration. Additionally, MSCs possess wide immunoregulatory properties via interaction with immune cells in both innate and adaptive immune systems, leading to immunosuppression of various effector functions. Their immunomodulatory functions are exerted by direct cell-to-cell contacts, secretion of cytokines and/or by a combination of both mechanisms.

The mechanisms of MSC interactions with the immune response are multifactorial and are exerted by direct cell-to-cell contacts, secretion of cytokines and/or by a combination of both mechanisms. The MSCs have the ability to interact with many kinds of immune cells, including B cells, T cells, dendritic cells (DCs), natural killer (NK) cells, neutrophil, and macrophages. On the other hand, the interactions that rely on cell-cell contact work depend on the secretion of soluble immune factors to induce MSC-regulated immunosuppression. These specific modulators, including a multitude of immune-modulatory factors, cytokines, and growth factors, modulate inflammatory responses and balance immune profiles.

Although MSCs, and in particular allogenic MSCs, have a great potential for cellular therapy in numerous diseases, the efficacy for tissue healing and/or disease outcome is not warranted as the host immune response rejects the cells. Multiple efforts have been made to improve MSC immunosuppressive potentials and prolong engraftment by for instance selecting specific MSCs.

US 2011 031 149 6 provides compositions of MSCs for promoting wound healing or fracture healing and methods of promoting wound healing or fracture healing comprising the steps of administering MSCs in an effective amount.

US 2014 001 778 7 provides an isolated, stimulated MSCs that selectively promote or suppress inflammation, as well as methods for producing and using the same. US' 787 uses stimulation of specific Toll-like receptors that affect immune modulating responses of MSCs to render an uniform preparation of cells for the improvement of stem cell based therapies.

EP 342 936 0 provides a method of selecting for MSCs possessing enhanced efficacy and donors whose MSCs possess an enhanced efficacy based on the expression of one or more markers.

EP 106 605 2 discloses a method of reducing an immune response to a transplant in a recipient by treating said recipient with an amount of MSCs effective to reduce or inhibit host rejection of the transplant. EP '052 focuses on inhibiting a T cell response to an alloantigen.

However there remains a need in the art for an improved immunomodulating MSC with a great therapeutic safety and efficacy.

The invention thereto aims to provide an isolated MSC characterized by specific immune-related properties in a representative inflammatory environment.

### SUMMARY OF THE INVENTION

The present invention provides an isolated MSC according to claim 1.

In a second aspect, the present invention provides a cell composition according to claim 7 comprising the isolated MSCs.

In a final aspect, the present invention provides the cell composition for use in the treatment of immune-related diseases and inflammatory processes according to claim 12.

### DESCRIPTION OF FIGURES

FIG. 1 shows a graphical representation of enzyme linked immune sorbent assay (ELISA) experiments, wherein an increased secretion of PgE2 by MSCs and PBMCs is evaluated.

FIG. 2 shows a graphical representation of ELISA experiments, wherein an increased expression TGF-β by MSCs is registered.

FIG. 3 shows a graphical representation of ELISA experiments, wherein an increased secretion of IL-6 by MSCs and PBMCs is evaluated.

FIG. 4 shows a graphical representation of ELISA experiments, wherein a decreased expression of TNF-α by PBMCs is visualized.

FIG. 5 visualizes the absence of MHC class II molecules on MSCs isolated from a horse.

### DETAILED DESCRIPTION OF THE INVENTION

The present invention concerns isolated MSCs with specific immunomodulating characteristics. More particularly, this invention concerns cell compositions comprising said isolated MSCs and the use thereof for the treatment of immune-related diseases and inflammatory processes.

The isolated MSCs of current invention possess immunomodulating properties, and are therefore desirable for cellular transplantation as immunogenic reactions of the host are circumvented.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

### Definitions

The term “isolated”, refers to both the physical identification and isolation of a cells from a cell culture or a biological sample, like blood, that can be performed by applying appropriate cell biology technologies that are either based on the inspection of cell cultures and on the characterization (and physical separation when possible and desired) of cells corresponding to the criteria, or on the automated sorting of cells according to the presence/absence of antigens and/or cell size (such as by FACS). In some embodiments, the terms “isolating” or “isolation” may comprise a further step of physical separation and/or quantification of the cells, especially by carrying out flow cytometry.

The term “mesenchymal stem cell” or “MSC” refers to multipotent, self-renewing cells that express a specific set of surface antigens and can differentiate into various cell types, including but not limited to adipocytes, chondrocytes, and osteocytes when cultured in vitro or when present in vivo.

As used herein, the “inflammatory environment” or “inflammatory condition” refers to a state or condition characterized by (i) an increase of at least one pro-inflammatory immune cell, pro-inflammatory cytokine, or pro-inflammatory chemokine; and (ii) a decrease of at least one anti-inflammatory immune cell, anti-inflammatory cytokine, or anti-inflammatory chemokine. Preferably the “inflammatory environment” or “inflammatory condition” as used herein comprises at least 15% proliferating T-lymphocytes, wherein said lymphocytes comprise at least T helper (Th)1 and Th2 cells and produce at least 7 pg per ml TNF-α and/or 13 pg per ml TGF-β.

The term “anti-inflammatory”, “anti-inflammation”, “immunosuppressive”, and “immunosuppressant” refers to any state or condition characterized by a decrease of at least one indication of localized inflammation (such as, but not limited to, heat, pain, swelling, redness, and loss of function) and/or a change in systemic state characterized by (i) a decrease of at least one pro-inflammatory immune cell, pro-inflammatory cytokine, or pro-inflammatory chemokine; and (ii) an increase of at least one anti-inflammatory immune cell, anti-inflammatory cytokine, or anti-inflammatory chemokine.

The term “peripheral blood mononuclear cells” or “PBMCs” of current invention comprise any peripheral blood cell, being lymphocytes (T-lymphocytes, B-lymphocytes and Natural Killer (NK) cells) and monocytes. Preferably at least 20.5% of said T-lymphocytes are positive for CD3 and/or at least 19.8% of said B-lymphocytes are positive for CD138 in current invention.

The term “positive” as used herein refers to the presence of biological activities and/or biological markers on the surface of the cell, intracellular, and/or secreted. Preferably a cell or a cell composition of current invention measures positive for a biological activity and/or marker, each for at least 60%, at least 70%, at least 80%, at least 90%, at least 95%, or at least 99%, or between 60% and 99%, or between 70% and 90%.

The term “negative” as used herein refers to the absence of biological activities and/or biological markers on the surface of the cell, intracellular, and/or secreted. Preferably a cell or cell composition of current invention measures negative for a biological activity and/or marker, each for less than 20%, less than 15%, less than 10%, less than 5%, or less than 2%.

The “population doubling time” or “PDT” of current invention is to be calculated by the formula: PDT=In(N_{f}/Nᵢ)/In(2), whereby N_{f} is the final number of cells after cell detachment and whereby Nᵢ is the initial number of cells at time point zero.

By the term “anti-coagulant”, it is meant a composition that can inhibit the coagulation of the blood. Examples of anticoagulants used in the present invention include EDTA or heparin.

The term “buffy coat” in this invention, is to be understood as the fraction of non-coagulated blood, preferably obtained by means of a density gradient centrifugation, whereby the fraction is enriched with white blood cells and platelets.

The term “blood-inter-phase” is to be understood as that fraction of the blood, preferably obtained by means of a density gradient, located between the bottom fraction, mainly consisting of erythrocytes and polymorphonuclear cells, and the upper fraction, mainly consisting of plasma. The blood-interphase is the source of blood mononuclear cells (BMCs) comprising monocytes, lymphocytes, and MSCs.

The term “suspension diameter” as used herein, is understood as the mean diameter of the cells, when being in suspension. Methods of measuring diameters are known in the art. Possible methods are flow cytometry, confocal microscopy, image cytometer, or other methods known in the art.

The terms “patient”, “subject”, “animal”, or “mammal” are used interchangeably and refer to a mammalian subject to be treated.

The term “inducing” or “induced” is to be understood as the process of activation of cell type specific genes or molecules in a multipotent or pluripotent cell, thereby driving such cell towards a more defined, specialized or differentiated cell lineage or cell type.

The term “therapeutically effective amount” is the minimum amount or concentration of a compound or composition that is effective to reduce the symptoms or to ameliorate the condition of a disease.

The term “treatment” refers to both therapeutic, prophylactic or preventive measures to reduce or prevent pathological conditions or disorders.

The term “in vitro” as used herein denotes outside, or external to, a body. The term “in vitro” as used herein should be understood to include “ex vivo”. The term “ex vivo” typically refers to tissues or cells removed from a body and maintained or propagated outside the body, e.g., in a culture vessel or a bioreactor.

### Description

The current invention is directed to a particular type of MSC, compositions comprising such MSCs and clinical use thereof.

In a first aspect, the invention provides an isolated MSC, wherein said cell measures positive for mesenchymal markers CD29, CD44 and CD90, and negative for MHC class II molecules. Said cell secretes the immunomodulatory PgE2 cytokine when present in an inflammatory environment or condition.

Inflammatory environments or conditions are characterized by the recruitment of immune cells of the blood. Inflammatory mediators include prostaglandins, inflammatory cytokines such as IL-1β, TNF-α, IL-6 and IL-15, chemokines such as IL-8 and other inflammatory proteins like TNF-α, IFN-γ. These mediators are primarily produced by monocytes, macrophages, T-cells, B-cells to recruit leukocytes at the site of inflammation and subsequently stimulate a complex network of stimulatory and inhibitory interactions to simultaneously destruct and heal the tissue from the inflammatory process.

Prostaglandin E2 (PgE2) is a subtype of the prostaglandin family. PgE2 is synthesized from arachidonic acid (AA) released from membrane phospholipids through sequential enzymatic reactions. Cyclooxygenase-2 (COX-2), known as prostaglandin-endoperoxidase synthase, converts AA to prostaglandin H₂ (PgH₂), and PgE₂ synthase isomerizes PgH₂ to PgE₂. As a rate-limiting enzyme, COX-2 controls PgE₂ synthesis in response to physiological conditions, including stimulation by growth factors, inflammatory cytokines and tumor promoters.

In a particular embodiment, said MSCs present in an inflammatory environment secrete the soluble immune factor prostaglandin E2 (PgE2) in a concentration ranging between 10³ to 10⁶ picogram per ml to induce MSC-regulated immunosuppression.

The PgE2 secretion of the MSCs in those specific concentration ranges stimulates anti-inflammatory processes in vitro and together with their ability to differentiate into appropriate cell types makes them desirable for cellular transplantation.

The isolated MSCs of current invention are characterized by the presence of mesenchymal markers CD29, CD44 and CD90. By means of the latter, the purity of the obtained MSCs can be analyzed, and the percentage of can be MSCs determined.

CD29 is a cell surface receptor encoded by the integrin beta 1 gene, wherein the receptor forms complexes with other proteins to regulating physiological activities upon binding of ligands. The CD44 antigen is a cell surface glycoprotein involved in cell-cell interactions, cell adhesion and migration. In addition, is CD44 a receptor for hyaluronic acid and can also interact with other ligands such as osteopontin, collagens and matrix metalloproteinases (MMPs). The CD90 antigen is a conserved cell surface protein considered as a marker for stem cells, like MSCs. The isolated MSC of current invention being triple positive for CD29/CD44/CD90 enables the person skilled in the art for a fast and unambiguous selection of the MSC and provides the MSC biological properties which are of interest for further downstream applications.

In a specific embodiment the MSCs of current invention measure positive for vimentin, fibronectin, Ki67, or a combination thereof which are typical MSC markers.

Furthermore, the isolated MSC of current invention is characterized by the absence of Major Histocompatibility Complex (MHC) Class II molecules, preferably all currently known MHC Class II molecules, classifying the cell as a cell that can be used in cellular therapy for mammalians, such as equine cellular therapy. Even when the isolated MSCs are partly differentiated, the MSCs remain negative for MHC class II molecules.

MSCs in general express MHC Class I antigen on their surface but limited amount of MHC Class II. In a particular embodiment the isolated MSC of current invention measures negative for MHC Class I marker as well. In a most preferred embodiment said MSC measures negative for MHC Class I and II markers, wherein said cell exhibits an extremely low immunogenic phenotype.

These immunological properties of the MSCs limits the ability of the recipient immune system to recognize and reject cells, preferably allogenic cells, following cellular transplantation. The production of factors by MSCs, that modulate the immune response together with their ability to differentiate into appropriate cell types under local stimuli make them desirable stem cells for cellular therapy.

In another and further embodiment the MSCs measure negative for CD45 antigen, a marker for hematopoietic cells.

In a most preferred embodiment the isolated MSC measures:
positive for mesenchymal markers CD29, CD44 and CD90;
positive for one or more markers comprised in the group consisting of vimentin, fibronectin, Ki67, or a combination thereof;
negative for MHC class I and/or II molecules; and
negative for hematopoietic marker CD45,
   wherein said cell secretes immunomodulatory PgE2 cytokine in a concentration ranging between 10³ to 10⁶ picogram per ml when present in an inflammatory environment or condition.

In general, any technology for identifying and characterizing cellular markers for a specific cell type (e.g. mesenchymal, hepatic, hematopoietic, epithelial, endothelial markers) or having a specific localization (e.g. intracellular, on cell surface, or secreted) that are published in the literature may be considered appropriate for characterizing MSCs. Such technologies may be grouped in two categories: those that allow maintaining cell integrity during the analysis, and those based on extracts (comprising proteins, nucleic acids, membranes, etc.) that are generated using such cells. Among the technologies for identifying such markers and measuring them as being positive or negative, immunocytochemistry or analysis of cell culture media are preferred since these allow marker detection even with the low amount of cells, without destroying them (as it would be in the case of Western Blot or Flow Cytometry).

Relevant biological features of the isolated MSCs can be identified by using technologies such as flow cytometry, immunocytochemistry, mass spectrometry, gel electrophoresis, an immunoassay (e.g. immunoblot, Western blot, immunoprecipitation, ELISA), nucleic acid amplification (e.g. real time RT-PCR), enzymatic activity, omics-technologies (proteomics, lipidomics, glycomics, transcriptomics, metabolomics) and/or other biological activity.

In a preferred embodiment, the MSC has an increased secretion of at least one of the molecules chosen of IL-6, IL-10, TGF-β, NO, or a combination thereof, and a decreased secretion of IL-1 when present in an inflammatory environment or condition. Comparison can be made with a mesenchymal stem cell having the same characteristics as presented above, but which is not subjected to said inflammatory environment or condition.

Preferably the MSC has an increased secretion of PgE2 in combination with two or more of the abovementioned factors.

PgE2, IL-6, IL-10, TGF-B and NO help inhibiting the proliferation and function of major immune cell populations like T cells and B cells. In addition the MSCs express low levels of MHC class I molecules and/or are negative for MHC class II molecules on their surface, escaping immunogenic reactions. In addition, the isolated MSCs of current can suppress the proliferation of white blood cells by their increased secretion of abovementioned factors, once again helping to avoid immunogenic reactions of the host.

The MSCs according to the present invention may originate from various tissues or body fluids, including but not limited to bone marrow, adipose, muscle, umbilical cord blood, peripheral blood, liver, placenta, skin, amniotic fluid. By preference, the MSCs originate from blood, more preferably peripheral blood. The MSCs of current invention may be derived by any standard protocol known in the art.

In an embodiment, said MSCs may be obtained via a method wherein the MSCs are isolated from blood or a blood phase and wherein said cells are cultured and expanded in a low glucose medium.

In an embodiment, such method may comprise the following steps:
a. the collection of one or more blood samples from donors, in a sample vial, coated with an anti-coagulant;
b. centrifuging the blood samples to obtain a 3-phase distribution, consisting of a plasma-phase, buffy coat, and erythrocytes phase;
c. collecting the buffy coat and loading it on a density gradient;
d. collecting of the blood-inter-phase obtained from the density gradient of step c);
e. isolating of MSCs from the blood-inter-phase by centrifugation;
f. seeding between 2.5×10⁵/cm² and 5×10⁵/cm² MSCs in culture and keeping them in a low glucose growth medium supplemented with dexamethasone, antibiotics and serum.

The number of seeding is crucial to ultimately obtain a pure and viable population MSCs at an acceptable concentration, as a too dense seeding will lead to massive cell death during expansion and a non-homogenous population of MSCs and a too dispersed seeding will result in little or no colony formation of MSCs, so that expansion is not or hardly possible, or it will take too much time. In both cases the viability of the cells will be negatively influenced.

In a preferred embodiment of current invention, the isolated MSCs have a high cell viability, wherein at least 90%, more preferably at least 95%, most preferably 100% of said cells are viable.

The blood-interphase is the source of blood mononuclear cells (BMCs) comprising monocytes, lymphocytes, and MSCs. By preference, the lymphocytes are washed away at 37° C., while the monocytes die within 2 weeks in the absence of cytokines necessary to keep them alive. In this way, the MSCs are purified. The isolation of the MSCs from the blood-inter-phase is preferably done by means of centrifugation of the blood-inter-phase, after which the cell pellet is washed at least once with a suitable buffer, such as a phosphate buffer.

In a further embodiment the isolated MSCs of current invention are negative for monocytes and macrophages, both within a range between 0% and 7.5%.

In particular, the mesenchymal cells are kept at least 2 weeks in growth medium. Preferably, growth medium with 1% dexamethasone is used, as the specific characteristics of the isolated MSCs are kept in said medium.

Following a minimum period of 2 weeks (14 days), preferably 3 weeks (21 days) MSC colonies will become visible in the culture bottles. In a subsequent step g) at least 6×10³ stem cells/cm² are transferred to an expansion medium containing low glucose, serum and antibiotics for the purpose of expanding the MSCs. Preferably, the expansion of the MSCs will occur in minimal five cell passages. In this way sufficient cells can be obtained. Preferably, the cells are split at 70% to 80% confluency. The MSCs can be maintained up to 50 passages in culture. After this the risk of loss in vitality, senescence or mutation formation occurs.

In a further embodiment, the population doubling time (PDT) between each passage during expansion of the isolated MSC should be between 0.7 and 3 days after trypsinization.

Said PDT between each passage during expansion of the isolated MSCs is preferably between 0.7 and 2.5 days after trypsinization.

In a preferred embodiment, the isolated MSC has a spindle-shaped morphology.

The morphological characterization of the isolated MSC of current invention classifies the cell as an elongated, fibroblast-like, spindle-shaped cell. This type of cell is distinct form other populations of MSCs with small self-renewing cells which reveal mostly a triangular or star-like cell shape and populations of MSCs with a large, cuboidal or flattened pattern with a prominent nucleus. The selection of MSCs with this specific morphological characteristic along with the biological markers enables the person skilled in the art to isolate the MSCs of current invention. A morphological analysis of cells can easily be performed by a person skilled in the art using phase-contrast microscopy. Besides, the size and granularity of MSCs can be evaluated using forward and side scatter diagram in flow cytometry or other techniques known by a person skilled in the art.

If desired, the isolated MSCs can be induced or differentiated towards adult cells. Induction or differentiation is preferably done by the addition of specific growth factors and/or other differentiation factors and/or inducing factors to the medium of the cells. The nature of these factors will crucially depend on the differentiation and the desired adult cell type. In a preferred embodiment, the MSCs according to the present invention can differentiate into tenocytes, chondrocytes, osteocytes, myocytes, adipocytes, or fibroblasts. In vitro differentiation results in an increase of expression of the MHC classes I and II. It is thus of relevance that our differentiation protocol showed no increase in these markers. While MHC class II expression was completely absent in MSCs, MHC class I was expressed at low levels in the MSCs.

In a more preferred embodiment, the MSC has a suspension diameter between 10 μm and 100 μm.

The isolated MSCs of current invention have been selected based on size/suspension diameter. By preference, the MSCs have a cell size between 10 to 100 μm, more preferably between 15 and 80 μm, more preferably 20 and 75 μm, more preferably between 25 and 50 μm. Preferably, the selection of cells based on cell size occurs by a filtration step. For instance, isolated MSCs with a cell concentration ranging between 10⁴ to 10⁷ MSCs per ml, wherein said cells are preferably diluted in low glucose DMEM medium, are selected by size by means of a filter system, wherein the cells are run through a double filtration step using a 40 μm filter. Double or multiple filtration steps are preferred. The latter provides for a high population of single cells and avoids the presence of cell aggregates. Such cell aggregates may cause cell death during the preservation of the cells by freezing and may all have an impact on further downstream applications of the cells. For instance, cell aggregates may higher the risk of the occurrence of a capillary embolism when administered intravenously.

In a further embodiment the isolated MSC induces the secretion of PgE2, IL-6, IL-10, NO, or a combination thereof and/or inhibits the secretion of TNF-α, IFN-γ, IL-1, or a combination thereof in PBMCs.

In the inflammatory environment the MSCs secrete multiple factors that modulate the immune response of the host. In addition, the MSCs have the stimulatory effect to induce the secretion of one or more factors selected from the group consisting of PgE2, IL-6, IL-10, NO, or a combination thereof. Next to the stimulatory effect of the MSCs on the PBMCs in an inflammatory environment, the MSCs also have an inhibitory effect on the secretion of the PBMCs, resulting in a decrease of one or more factors selected from the group consisting of TNF-α, IFN-γ, IL-1, or a combination thereof. The MSCs have a regulatory effect in the inflammatory environment, making them useful in the treatment of all sorts of diseases, particularly disorders of the immune system.

In a preferred embodiment the immunomodulatory activities of the MSCs in the inflammatory environment are optimal when MSCs and PBMCs are present in a ratio ranging between 1:0.001 to 1:1000. A particular preferred ratio between MSCs and PBMCs is 1:500, more preferably 1:100, more preferably 1:10.

In a particular preferred embodiment, the MSC is isolated from blood, preferably peripheral blood. Blood appears to be an optimal source of MSCs. Blood is not only a non-invasive and painless source, but also simple and safe to collect and, consequently, easily accessible. The blood may originate from all mammals, especially horse, human, cat, dogs, rodents, etc. By preference, said origin of is equine.

In a second aspect, the invention provides a cell composition comprising at least 60% of the isolated MSCs of current invention, wherein at least 95% of said cells are single cells.

By preference, said composition according to the present invention comprises at least 90% MSCs. More preferably, the cell composition comprises at least 95% MSCs, more preferably at least 99%. In an embodiment, said cell composition is a pure composition, comprising 100% MSCs according to the current invention.

In a preferred embodiment, said composition comprises at least 75%, more preferably at least 80%, even more preferably at least 85%, most preferably at least 90% of single cells. Preferably said cells have a suspension diameter of between 10 μm and 100 μm, the single-cell nature of the cells and the diameter of the cells is crucial for any downstream application, like cellular therapy, and for the vitality of the cells.

In a more preferred embodiment of the composition, the isolated MSCs measure positive for CD29 in a range between 95% and 100%, CD90 in a range between 95% and 100%, and CD44 in a range between 80% and 100%, more preferably between 90% and 100%, most preferably between 95% and 100%, and measures negative for MHC Class II molecules in a range between 0% and 5%, more preferably 0% and 2%. Furthermore, the MSC measures negative for MHC Class I molecules in a range between 0% and 60%, more preferably between 0% and 50%, more preferably between 0% and 45%. In a further embodiment of the composition, the MSCs measure negative for CD45 within a range between 0% and 7.5%.

In a more preferred embodiment, the MSCs comprised in the composition have a cell viability of at least 90%, more preferably 95%, more preferably 99%, more preferably 100%.

Another embodiment of current invention relates a cell composition obtained by differentiation of the MSCs composition according to any of the previous embodiments, whereby the cells of said cell composition are tenocytes, chondrocytes, osteocytes, myocytes, adipocytes, keratinocytes, neurons or fibroblast.

In a preferred embodiment the cell composition comprises a therapeutically effective amount of isolated MSCs, preferably said composition comprises 10⁵ to 10⁷ of said isolated MSCs per ml.

The minimum therapeutically effective dose that yields a therapeutic benefit to a subject is at least 10⁵ of the isolated MSCs per ml. Said MSCs may be diluted in the cell composition. Preferably, the cell composition comprises between 10⁵ to 5×10⁵ isolated MSCs per ml.

In another embodiment the cell composition can be complemented with used with components selected from the group consisting of platelet-rich plasma (PRP), hyaluronic acid, compositions based on hyaluronic acid, glycosaminoglycans, or compositions based on glycosaminoglycans. Mixing of the composition with such carrier substances may in some cases be desirable to increase the effectiveness of the composition or create a synergistic effect. For instance, said carrier substances aid in the homing capacities and immunomodulating effects of the MSCs in the cell composition.

In a further embodiment of the cell composition, the isolated MSCs express PgE2, IL-6, IL-10, TGF-β, NO or a combination thereof when in the presence of PBMCs.

In a preferred embodiment of the cell composition, the isolated MSCs induce the expression of pgE2, IL-6, IL-10, NO, or a combination thereof in PBMCs and/or inhibit the secretion of TNF-α, IFN-γ, IL-1, or a combination thereof in PBMCs, when in the presence of PMCs

The isolated MSCs keep their biological capacities to secrete immunomodulating factors, in the presence of PBMCs. The immune response mediated by the PBMCs after the MSCs' stimulatory effect, like the secretion of PgE2 or other factors is pivotal for the MSCs to further exert immunosuppressive action.

In addition, the ratio of the MSCs and PBMCs is a crucial factor. Therefore, a more preferred embodiment of the cell composition concerns the presence of MSCs and PBMCs in a ratio of between 1:0.001 and 1:1000. Said ratio of MSCs and PBMCs is preferably 1:500, more preferably 1:100, more preferably 1:10.

In vitro tests have shown that these ratios result in an efficient immunomodulation of the MSCs in an inflammatory environment. Same results are expected in vivo, resulting in an efficient and safe cellular therapy.

In another and further embodiment, the cell composition, when in the presence of PBMCs, will express PgE2 at a concentration of between 10³ to 10⁶ picogram per ml.

The concentration of 10⁵ to 10⁷, more preferably 10⁵ to 10⁶, of isolated MSCs per ml in the presence of PBMCs in said cell composition is marked by the secretion of the immunosuppressive factor PgE2, wherein said amount of cells secrete PgE2 in a range between 10³ to 10⁶ picogram per ml. Preferably 10⁴ to 10⁵ picogram per ml PgE2 is secreted by the MSCs in the cell composition to sufficiently restrain the immunocompetent cells.

In a preferred embodiment, the cell composition according to the invention is formulated for administration in a subject by means of intravenous, intraarticular, intramuscular, intralesional, intraarterial, topical, subconjunctival injection or regional perfusion.

In a particular embodiment the abovementioned compositions may be used for allogenic administration to a subject. Allogenic use allows a better control of the quality of the MSCs, as different donors may be screened, and the optimal donors may be selected. In view of preparing functional MSCs, the latter is indispensable. This is in contrast to autologous use of MSCs, as in this case, quality of the cells cannot be ensured.

For instance when blood MSCs were isolated, blood from a donor was used who was later also recipient of his isolated MSCs. In another embodiment, blood is used from donors in which the donor is preferably of the same family, gender or race as the recipient of the MSCs isolated from the blood of donors. In particular, these donors will be tested on common current transmittable diseases or pathologies, in order to avoid the risk of horizontal transmission of these pathologies or diseases through the stem cells. Preferably, the donor animals are kept in quarantine. When using donor horses they can be, for example tested for the following pathologies, viruses or parasites: equine infectious anemia (EIA), equine rhinopneumonitis (EHV-1, EHV-4), equine viral arteritis (EVA), West Nile virus (WNV), African horse Sickness (AHS), dourine (Trypanosoma), equine piroplasmosis, glanders (malleus, glanders), equine influenza, Lyme borreliosis (LB) (Borrelia burgdorferi, Lyme disease).

The composition according to the current invention will by preference be frozen in order to allow long-time storage of the composition. Preferably the composition will be frozen at low and constant temperature, such as a temperature below −20° C. These conditions allow a save storage of the composition, and enable the cells in the composition to keep their biological and morphological characteristics, as well as their high cell viability during storage and once thawed.

In a more preferred embodiment the cell composition can be stored for at least 6 months at a maximum temperature of −80° C., optionally in liquid nitrogen.

A crucial factor in the freezing of the MSCs is the composition of the cryogenic medium, in particular, the concentration of DMSO. DMSO prevents ice crystal formation in the medium during the freezing process, but may be toxic to the cells in high concentrations. In a preferred embodiment, the concentration of DMSO comprises up to 20%, more preferably up to 15%, more preferably the concentration of DMSO in the cryogen comprises 10%. The cryogenic medium further comprises low-glucose medium such as low glucose DMEM (Dulbecco's Modified Eagle Medium).

Afterwards, the cell composition is preferably thawed before administration at a temperature around room temperature, preferably at a temperature between 20° C. and 37° C., more preferably at a temperature between 25° C. and 37° C., and in a time span of maximal 20 minutes, preferably maximal 10 minutes, more preferably maximal 5 minutes.

Furthermore, the composition is administered within 2 minutes after thawing, in order to safeguard the vitality of the composition.

By preference, the current invention has its application in the veterinary field. The composition can be administered to a subject, whereby said subject is a mammal, preferably a dog, cat, horse, or monkey.

In a third aspect, the invention provides the cell composition of current invention for use in the treatment of immune-related illnesses and/or inflammatory processes in a subject, preferably a mammalian subject.

In particular said immune-related illnesses are selected from the group consisting of auto-immune disease, atopic dermatitis, allergies, rheumatoid arthritis, and arthritis; and said inflammatory processes are selected from the group consisting of degenerative joint disease, osteoarthritis, fever, lung asthma, and tendinitis.

In a particular preferred embodiment of current invention, the cell composition is used in a treatment as listed above, wherein per treatment 10⁵ to 10⁷ of said isolated MSCs are administered, wherein said administered preferably by means of intravenous, intraarticular, intramuscular, intralesional, intraarterial, topical subconjunctival injection or regional perfusion.

The invention is further described by the following non-limiting examples which further illustrate the invention, and are not intended to, nor should they be interpreted to, limit the scope of the invention.

### EXAMPLES

It is supposed that the present invention is not restricted to any form of realization described previously and that some modifications can be added to the presented example of fabrication without reappraisal of the appended claims.

### Example 1: Quantification of PgE2 Secretion by the Isolated MSCs by an Immunoassay Technique, ELISA

Equine peripheral blood mononuclear cells (ePBMCs) were obtained by collecting 50 ml of peripheral blood into a tube with ethylenediaminetetraacetic acid (EDTA) as an anticoagulant from a horse with a chronic inflammation of the tendon. The EDTA tubes were centrifuged for 20 minutes, followed by the collection of the buffy coat into a sterile 15 ml tube. The buffy coat was two times diluted with phosphate buffer, 1× PBS. The solution was subsequently treated with Percoll (1.35 g/ml) and centrifuged for 15 minutes. Next, the interphase was collected and washed 1× PBS by centrifugation for 8 minutes, and repeated twice. The pellet was resuspended and cells were counted. After counting PBMCs were first labeled, seeded in 96-well plates and grown in growth medium containing beta mercapto-ethanol in the presence of MSCs or not. 25×10⁶ ePBMCs were seeded per flask (T75 flask) in a specific ratio with MSCS, wherein said ratio of MSCS and PBMCs is 1:10. Supernatants from MSCs and PBMCs was obtained after 96 hours of incubation and used for immunoassay. Next to the test sample, comprising stimulated PBMCs and MSCs, a positive and negative control was evaluated for the expression of PgE2 secretion.

PgE2 secretion was quantified using enzyme-linked immunosorbent assay ELISA kit competitive ELISA (Enzo Life Sciences, Farmingdale, NY, USA). An ELISA plate was coated with anti-equine PgE2 monoclonal antibody overnight, washed, and incubated with the samples, test and control samples. The plate was washed again, incubated with anti-equine PgE2 biotin-labeled monoclonal antibody, washed, incubated with avidin, horseradish peroxidase, washed again, incubated with peroxidase substrate, and read at 405 nm on a plate reader.

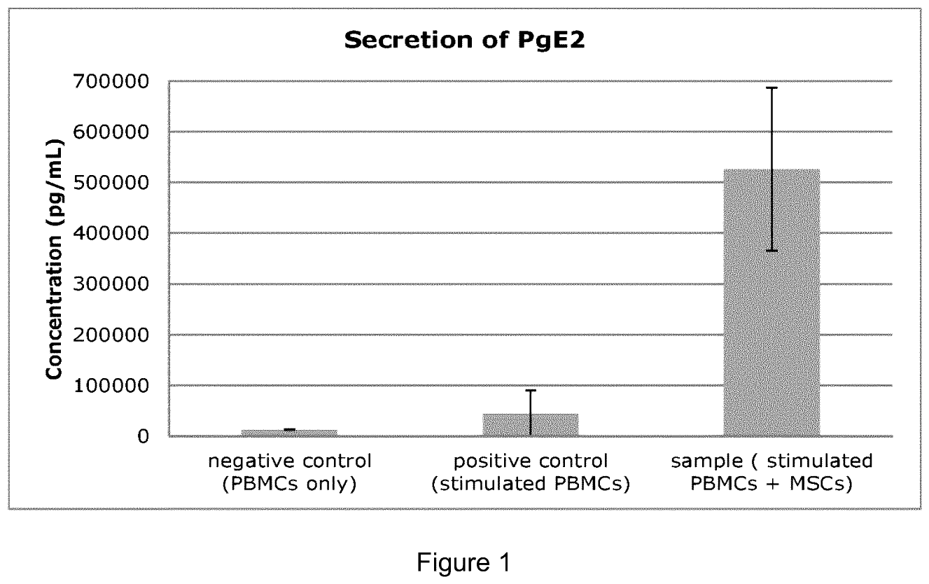

As visualized in FIG. 1 the secretion of PgE2 in MSCs is induced in an inflammatory environment as an increase of more than 500,000 pg/ml cells is evaluated.

### Example 2: Quantification of TGF-β and IL-6 Secretion by MSCs and PBMCs by ELISA

TGF-β and IL-6 secretion was quantified using a competitive ELISA kit (Cusabio, USA). The competitive inhibition enzyme immunoassay technique follows the same principles as the PgE2 competitive ELISA kit as previously described in example 1.

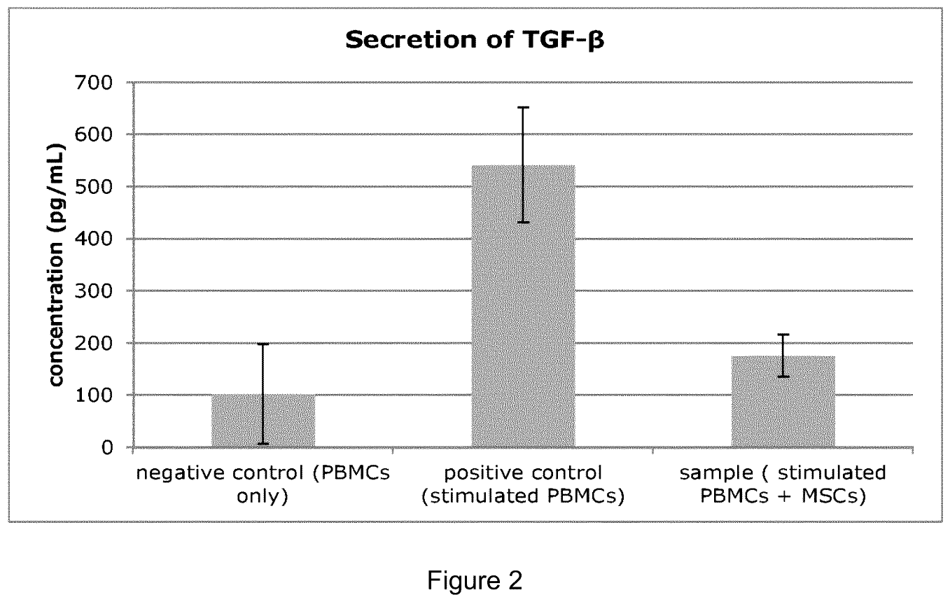

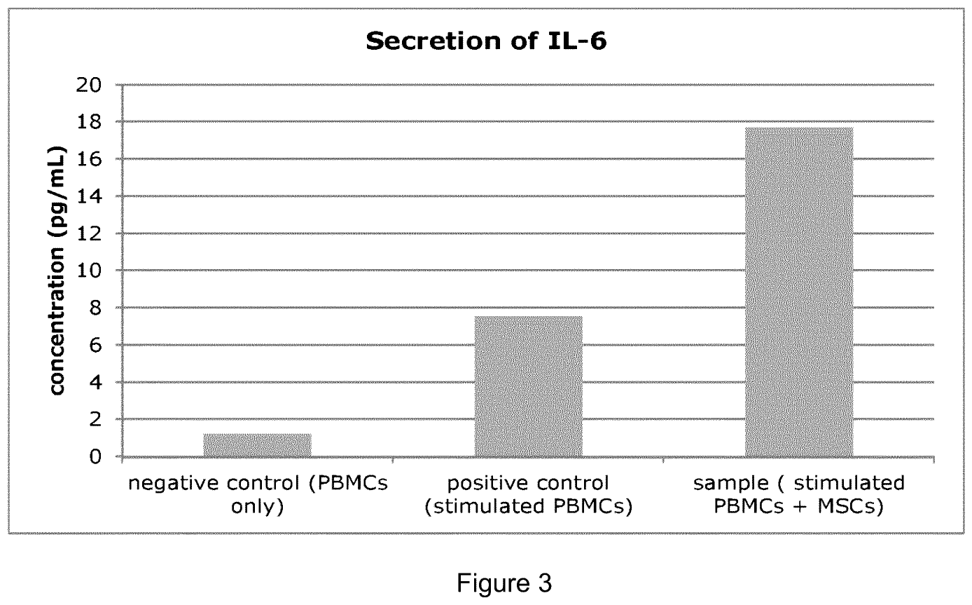

An increased secretion of TGF-β and IL-6 by MSCs is monitored, as well as an increased secretion of IL-6 in PBMCs. FIG. 2 visualize the increased secretion of TGF-β by MSCs with almost a doubling concentration of TGF-β. The secretion of IL-6 by MSCs and PBMCs is almost eighteen times higher than the negative control, as visualized in FIG. 3.

### Example 3: Quantification of TNF-α Expression by PBMCs by ELISA

TNF-α expression was measured using a competitive ELISA kit (Invitrogen, USA). An ELISA plate was coated with anti-equine TNF-α monoclonal antibody overnight, washed, and incubated with the samples, test and control samples. The plate was washed again, incubated with anti-equine PgE2 biotin-labeled monoclonal antibody, washed, incubated with streptavidin-horseradish peroxidase, washed again, incubated with peroxidase substrate, and read at 405 nm on a plate reader.

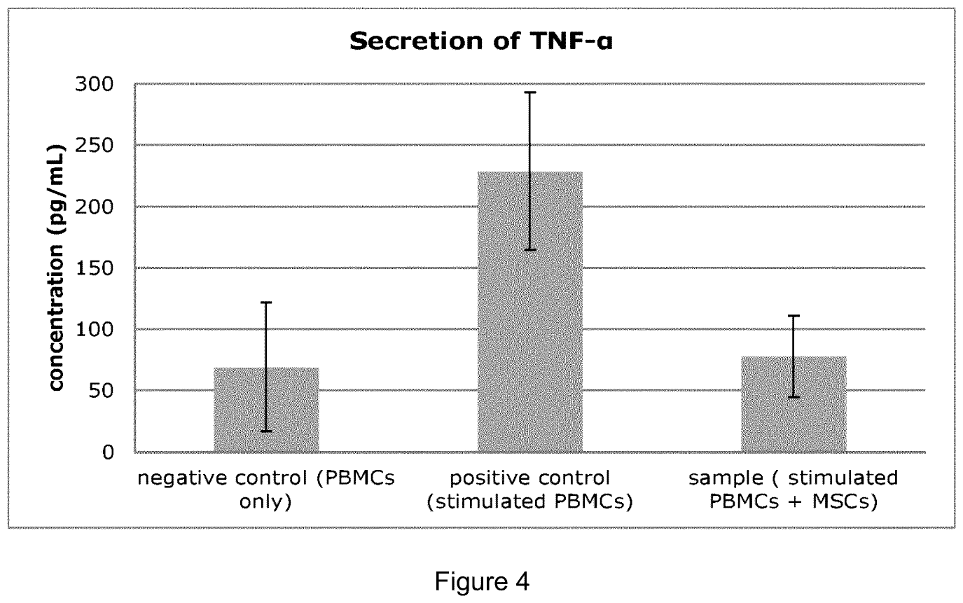

A decrease in secretion of TNF-α by PBMCs was quantified when in the presence of MSCs. The regulatory effect of the MSCs inhibits the TNF-α secretion of the PBMCs with a third, as a decrease of 231 pg/ml to 77 pg/ml is quantified, as visualized in FIG. 4.

### Example 4: Immunophenotypic Characterization of MSCs by Flow Cytometry

The expression of several stem cell markers was evaluated by flow cytometry to characterize the MSCs immunophenotypically. The expression of the typical rejection proteins, major histocompatibility complex (MHC) class II was evaluated on native MSCs and MSCs in an inflammatory environment. Per series, 4×10⁵ cells were used and labeled with the primary antibodies mouse anti-horse MHC class II IgG1 (Abd Serotec, 1:50). Cells were incubated with the primary antibodies for 15 minutes on ice in the dark and washed twice in washing buffer, consisting of DMEM with 1% bovine serum albumin (BSA). A secondary rabbit anti-mouse-FITC (Abcam, 1:100) antibody was used to identify positive cells after 15 minutes of incubation on ice in the dark. Finally, all cells were washed three times in washing buffer and at least 10³ cells were evaluated using a fluorescence activated cell sorter (FACS) Canto flow cytometer (Becton Dickinson Immunocytometry systems) equipped with a 488 nm solid state and a 633 nm HeNe laser, and these data were subsequently analyzed with the FACS Diva software. All analyses were based on (i) autofluorescence and (ii) control cells incubated with isotype-specific IgG's, in order to establish the background signal. All isotypes were matched to the immunoglobulin subtype and used at the same protein concentration as the corresponding antibodies.

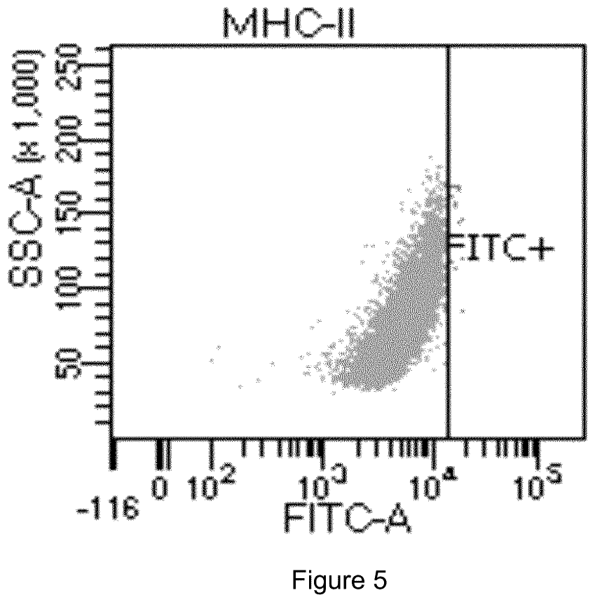

The native (data not shown) MSCs and MSCs in an inflammatory environment (see FIG. 5) are negative for MHC class II molecules.

1. A method comprising administering to an individual in need thereof a composition for treatment of an immune-related disease and/or an inflammatory process in the individual, the composition comprising isolated mesenchymal stem cells (MSCs), wherein said cells are measured positive for mesenchymal markers CD29, CD44 and CD90 and negative for MHC class II molecules,
wherein said cells have been isolated from an individual and placed into an inflammatory condition or environment and secretes immunomodulatory prostaglandin E2 cytokine in the inflammatory environment or condition,
wherein the secretion of the prostaglandin E2 cytokine is more than 500,000 pg/ml relative to secretion of prostaglandin E2 cytokine measured from control mesenchymal stem cells isolated from the same individual but not placed in the inflammatory environment or condition,
wherein the composition comprises at least 60% of the isolated MSCs, and
wherein at least 95% of said cells are single cells.

2. The method of claim 1, wherein 10⁵ to 10⁷ of said isolated MSCs are administered, wherein administration is by intravenous, intraarticular, intramuscular, intralesional, intraarterial, topical, subconjunctival injection or regional perfusion administration.

3. The method of claim 1, wherein the individual has the immune-related disease that is selected from the group consisting of atopic dermatitis, allergies, rheumatoid arthritis, and arthritis; or wherein the individual has the inflammatory processe that is selected from the group consisting of degenerative joint disease, osteoarthritis, fever, lung asthma, and tendinitis.
